(19) United States
(12) Patent Application Publication
(10) Pub. No.: US 2006/0004301 A1
(43) Pub. Date: Jan. 5, 2006

(54) Clinical application of electrical impedance tomography to characterize tissue

(76) Inventor: Raymond S. Kasevich, Great Barrington, MA (US)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/159,822

(22) Filed: Jun. 23, 2005

Related U.S. Application Data
(60) Provisional application No. 60/582,720, filed on Jun. 24, 2004.

(51) Int. Cl. A61B 5/05

(52) U.S. Cl. 600/547

(57) ABSTRACT

The present invention is a system for use in creating images of portions of human tissue inside the body by electrical impedance tomography. The system may comprise at least one flexible tube for insertion in the body in proximity to a targeted portion of human tissue in the body, an inflatable balloon removably attached to the distal end of the flexible tube, a first array of electrodes attached to the surface of the inflatable balloon for at least one of injecting current into the targeted portion of human tissue and receiving current that was injected into the targeted portion of human tissue, and a second array of electrodes for at least one of injecting current into the targeted portion of human tissue to the first array of electrodes and for receiving current from the first array of electrodes.

### CROSS-REFERENCE TO RELATED APPLICATION(S)

This application claims priority from U.S. provisional patent application No. 60/582,720, which was filed on Jun. 24, 2004, and which is incorporated herein by reference in its entirety.

### FIELD OF THE INVENTION

The present invention generally relates to the use of electrical impedance tomography (“EIT”) to characterize tissue in the human body. More particularly, this invention relates to systems for using EIT inside the human body to create high quality images of tissue to assist in the diagnosis and treatment of disease.

### BACKGROUND OF THE INVENTION

There is a widespread need for high-quality images of human tissue in connection with diagnosing and treating diseases and other conditions. To name just a few examples, such imagery is invaluable for locating and treating tumors, diagnosing and locating pulmonary emboli, diagnosing and treating heart disease, and monitoring blood volume and blood flow.

Traditional systems for creating images of human tissue for medical purposes include x-rays, computerized axial tomography (“CAT scans”), magnetic resonance imagery (“MRI”), and ultrasound. Such systems are capable of creating very detailed images. However, each presents certain disadvantages as well. For example, systems such as x-rays and CAT scans expose the body to potentially harmful radiation. Scanning with MRI may be dangerous or uncomfortable for certain patients, and additionally is quite expensive. Many of the traditional systems are complex.

EIT presents an alternative method for imaging human tissue. EIT produces images of the resistivity, or impedance, within the tissue. Although systems such as MRI and CAT scans create higher quality images, EIT is substantially less expensive and less complex than those systems. In addition, EIT does not expose the patient to radiation or other harmful effects, and thus is safe and suitable for long-term monitoring of the patient. Further, EIT is the most effective method of monitoring certain functions, such as blood volume and blood flow.

To date, almost all research and application of EIT in a clinical setting has been done using electrodes placed on the outside of the body. The images created by EIT, however, would be improved through the use of a system that utilized some, or all, of the electrodes inside the body. Thus, there is a need for systems that generate EIT images of human tissue using some or all of the electrodes inside the body.

### BRIEF SUMMARY OF THE INVENTION

Electrical impedance tomography is a relatively new technique for clinical applications that involve the measurement of some property within the body which causes a corresponding change in electrical resistivity. The use of EIT in clinical applications is based on the fact that different types of human tissue have different electrical resistivities. For example, the resistivity of human blood is approximately 15 Ohms per cm, whereas that of lung tissue is approximately 2000 Ohms per cm. Furthermore, certain conditions, such as the application of heat, cause a corresponding change in the electrical resistivity with human tissue. By applying voltages or currents to the electrode arrays, one can measure the resistivity of the tissue. That data then may be used to create an image of the tissue.

EIT has several promising applications in the clinical setting. For example, the resistivity of tumors typically differs dramatically from that of the surrounding tissue; thus EIT may be used to locate, and create images of, such tumors. Similarly, EIT may be used to visualize blood perfusion in the heart and respiratory function. Because EIT can measure changes in temperature in human tissue, it also may be used to monitor hyperthermia or thermotherapy treatments.

EIT clinical applications generally involve electrodes employed outside the body, electrically attached to the skin. EIT would be improved, however, through the use of a system that allowed the use some or all of the electrodes inside the body in proximity to the targeted tissue. The present invention is such a system.

In one embodiment of the present invention, a system may be provided for use in creating images of portions of human tissue inside the body by electrical impedance tomography. The system may comprise at least one flexible tube for insertion in the body in proximity to a targeted portion of human tissue in the body. One embodiment of the system may include an inflatable balloon removably attached to the distal end of the flexible tube, a first array of electrodes attached to the surface of the inflatable balloon for at least one of injecting current into the targeted portion of human tissue and receiving current that was injected into the targeted portion of human tissue, and a second array of electrodes for at least one of injecting current into the targeted portion of human tissue to the first array of electrodes and for receiving current from the first array of electrodes.

In one embodiment of the present invention, a system may be provided for use in creating images of the prostate gland by electrical impedance tomography. The system may comprise at least one flexible tube for insertion in the body in proximity to the prostate gland, an inflatable balloon removably attached to the distal end of the flexible tube, a first array of electrodes attached to the surface of the inflatable balloon for at least one of injecting current into the prostate gland and receiving current that was injected into the prostate gland, and a second array of electrodes for at least one of injecting current into the prostate gland to the first array of electrodes and for receiving current from the first array of electrodes.

In one embodiment of the present invention, a method may be provided for creating images of portions of human tissue inside the body by electrical impedance tomography. The method may comprise the steps of inserting in the body in proximity to a targeted portion of tissue a first flexible tube with a first inflatable balloon removably attached to the distal end, such first inflatable balloon having first array of electrodes attached to its surface for at least one of injecting current into the targeted portion of human tissue and for receiving current that was injected into the targeted portion of human tissue, placing in proximity to the targeted portion of tissue a second array of electrodes for at least one of injecting current into the targeted portion of human tissue to the first array of electrodes and for receiving current from the first array of electrodes, injecting a current into the targeted portion of human tissue with the first or second array of electrodes, and receiving the current with the second or first array of electrodes.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a perspective view of an embodiment of the present invention incorporating two electrode arrays attached to inflatable balloons inserted into the body with a flexible tube on either side of the targeted tissue.

FIG. 2 is a perspective view of an embodiment of the present invention incorporating one electrode array attached to an inflatable balloon inserted into the body in the proximity of the targeted tissue with a flexible tube and another electrode array attached to the outside of the body.

FIG. 3 is a perspective view of an embodiment of the present invention incorporating two electrode arrays attached to a single inflatable balloon inserted into the body in the proximity of the targeted tissue with a flexible tube.

FIG. 4 is a perspective view of an embodiment of the present invention in which the system is used to create images of the prostate gland during treatment by a microwave antenna.

FIG. 5 is a representation of a possible array of electrodes wrapped around a balloon surface.

### DETAILED DESCRIPTION

The present invention is a system and method for using electrical impedance tomography to characterize tissue in the human body. Any such system requires at least two sets of electrodes, one of current injection electrodes and one of current return electrodes. Voltages and currents may be applied to the electrode arrays, which creates a current from one to the other that runs through the intervening tissue. The system permits a measurement of the resistivity of the intervening tissue, which measurements are then used to create an image of the tissue which can be used to diagnose and/or treat disease or other conditions.

In the system and method of the current invention, one or both of the sets of electrodes are located inside the body during operation. For example, in one embodiment, the current injection electrode array is attached to the exterior of an expandable balloon. The expandable balloon is removably attached to the end of a flexible tube, such as a catheter, that can be inserted in the body through a blood vessel or other cavity. Alternatively, the electrodes may be imprinted on a catheter or other slender structure that can be inserted in the body. The current return electrode array may rest either inside or outside the body. For example, in one embodiment, the return electrode array may be attached to the exterior of the same expandable balloon to which the current injection array is attached. In another embodiment, the injection return array may be attached to a second flexible tube and inserted into the body. In this embodiment, both electrode arrays are placed in proximity to the targeted tissue and on roughly opposite sides of the tissue. The balloons are expanded to ensure contact between the electrodes and the tissue. A current is then generated and runs between the electrode arrays. The resistivities of the tissue are measured, and then used to generate an image of the targeted tissue. In yet another embodiment, the current return array may be attached to the exterior of the body.

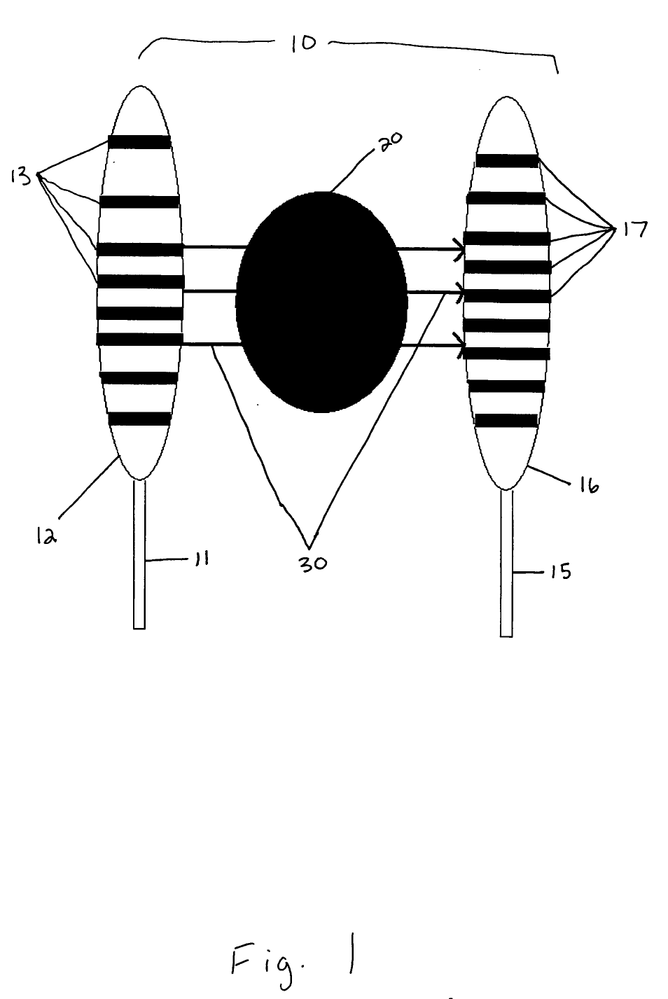

FIG. 1 is a perspective view of an embodiment of the present invention in which both the electrode arrays are inserted into the body on roughly opposite sides of the targeted tissue. The system 10 comprises current injection electrodes 13 arranged in an array and attached to expandable balloon 12. The expandable balloon 12 is removably attached to flexible tube 11, which is used to insert the balloon and electrode array inside the body in proximity to the targeted tissue 20. Similarly, current return electrodes 17 are arranged in an array and attached to expandable balloon 16. The expandable balloon 16 is removably attached to flexible tube 15, which is used to insert the balloon and electrode array inside the body, in proximity to the targeted tissue 20 and on the opposite side from expandable balloon 12. In one embodiment, after both balloons are inflated, a current generator may be attached to the electrodes 13 and 17 and used to generate a current 30 that runs between the electrodes 13 and the electrodes 17, running through the targeted tissue 20. An image generator also may be used to measure the resistivities of the targeted tissue 20 and to create an image of that tissue.

The current injection and current return electrode arrays may consist of a broad range of number of electrodes. Even a single current injection electrode and single current return electrode may provide very limited resistivity data. However, increasing the number of electrodes will result in improvement in the spatial resolution of the image created. For example, if N represents the number electrodes, 2N will result in 4 times more measurements of resistivities than N electrodes, thus doubling the spatial resolution. The number of electrodes that may be used will be limited by the physical space on which the electrodes must be placed. Those skilled in the art will be familiar with the limits on the number of electrodes and the proper spacing of electrodes that may be used in they system of the invention.

The electrodes 13 and 17 may be made from a variety of materials known in the art. For example, in one embodiment of the present invention, the electrodes 13 and 17 may be silver electrodes, silver-chloride coated electrodes, tin electrodes, tin-chloride coated electrodes, stainless steel electrodes, carbon electrodes, conductive plastic electrodes, or combinations of those. Those skilled in the art will be familiar with a variety of electrodes that may be used for the present invention. It is understood that such electrodes should be non-toxic and safe for use in the human body. In one embodiment of the present invention, the electrodes 13 and 17 may be designed to minimize interference with electromagnetic wave energy so as to facilitate their use in conjunction with heat treatment utilizing radiofrequency or microwave energy.

The electrodes 13 and 17 may be attached to the outside or the inside of the body using material to lower the impedance of the connection. For example, in one embodiment of the present invention, materials such as a saline gel or karaya gum may be used to facilitate the connection between the electrodes 13 and 17 and the interior or exterior body surface and lower the impedance of that connection. Saline gels for use with the present invention may range from 0.5 percent sodium chloride to 20% sodium chloride. Such a gel with 4% sodium chloride will result in a lower impedance than that provided by sea water; saline levels in excess of 20% sodium chloride may result in irritation to the body surface. Those skilled in the art will understand that a variety of such materials are known in the art. It is understood that one of the advantages of the present invention is that impedances of the connection between the electrodes 13 and 17 and the body surface are naturally lower inside the body.

In one embodiment of the present invention, expandable balloons 12 and 16 may be standard expandable balloons known in the art, such as balloons used with balloon catheters to perform angioplasty procedures. Those skilled in the art will understand that such balloons may be made from a variety of materials and may be designed in a variety of shapes. In another embodiment of the present invention, flexible tubes 11 and 15 may be standard catheters, such as those used in angioplasty procedures. Again, those skilled in the art will understand that such catheters may be made of a variety of materials and to a variety of specifications. Flexible tubes 11 and 15 with expandable balloons 12 and 16 may be inserted into the body in a variety of fashions. For example, in one embodiment, such tubes and balloons may be inserted in the body and advanced to the desired location through blood vessels. In another embodiment, the tubes and balloons may be inserted in the body and advanced to the desired position through the rectum or urethra. It is understood by those skilled in the art that such tubes and balloons may be inserted in the body through any vessel that is large enough to accommodate them.

Those skilled in the art know that a range of currents may be injected into the targeted area. Such current may range from 0.5 to 5 milliamps. It is understood that the current injected should not exceed the maximum safe level. Those skilled in the art will understand that current levels above 5 milliamps may be dangerous to humans. The range of frequencies used may range from 15 KHz to 1 MHz. In one embodiment of the present invention, hardware is incorporated into the system to limit the current and frequency that may be applied so as to ensure safety.

Those skilled in the art will understand that the image created by the system may be improved by dynamic beam steering. In one embodiment of the present invention, balloons 12 and 16 may be shifted or rotated mechanically to direct the electrical currents, thereby allowing focus on particular areas and improving the image created. In another embodiment, the user may reprogram the frequency, amplitude, or other characteristics of the injection current or voltages to improve the image quality.

The present invention may be used to create images of a variety of areas of the body. For example, in one embodiment, the system may be used to create images of the prostate gland to diagnosis prostate abnormalities, such as prostate cancer. In one embodiment, they present invention may be used to create images of the prostate gland during treatment, such as hyperthermia treatment, to monitor such treatment. In another embodiment, the present invention may be used to create images of the lung to assist in the diagnosis and treatment of conditions such as lung cancer and pulmonary embolisms. In yet another embodiment, the present invention may be used to make images of the heart and to monitor such body functions as blood flow and blood volume. In another embodiment, the present invention may be used to create images of breast tissue to assist in the diagnosis and treatment of conditions such as breast cancer. Those skilled in the art will understand that the present invention has a variety of other applications for creating images of portions of the human body.

A variety of devices to generate the current required in the present system may be used. For example, either direct current generators or alternating current generators may be used. In another embodiment, a battery may be used to generate current. Those skilled in the art will be familiar with a variety of current generators that may be used in conjunction with present invention.

Devices for calculating the resistivity of tissue and using algorithms to reconstruct the image of the targeted tissue are well-known in the art. For example, the calculation of resistivity and reconstruction of images based on those measurements are both described in detail in the following references: K. Boone, et al., “Imaging with Electricity: Report of the European Concerted Action on Impedance Tomography,” Journal of Medical Eng'g & Tech., vol. 21, no. 6 (November/December 1997), pages 201-232; and Isaacson, David, “Distinguishability of Conductivities by Electric Current Computed Tomography,” IEEE Transactions on Medical Imaging, vol. MI-S, no. 2, June 1986, pages 91-95. Those references are hereby incorporated herein in their entirety.

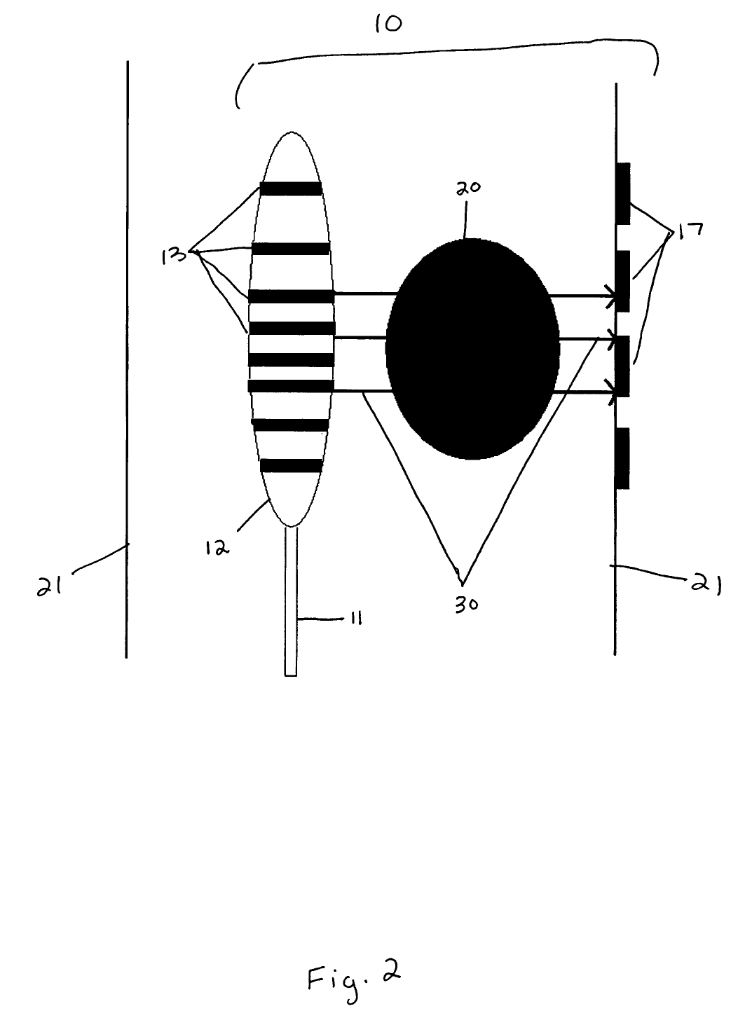

FIG. 2 is a perspective view of an embodiment of the present invention in which one electrode array is inserted into the body in proximity to the targeted tissue and a second electrode array is attached to the outside of the body. The system 10 comprises current injection electrodes 13 arranged in an array and attached to expandable balloon 12. The expandable balloon 12 is removably attached to flexible tube 11, which is used to insert the balloon and electrode array inside the body in proximity to the targeted tissue 20. Current return electrodes 17 are arranged in an array and attached directly to the exterior of the body 21. After expandable balloon 12 is inflated, a current generator, which is attached to the electrode arrays 13 and 17, is used to generate a current 30 that runs between the electrodes 13 and the electrodes 17 and runs through the targeted tissue 20. A generator, such as a computer running appropriate software, measures the resistivities of the targeted tissue 20 and creates an image of that tissue.

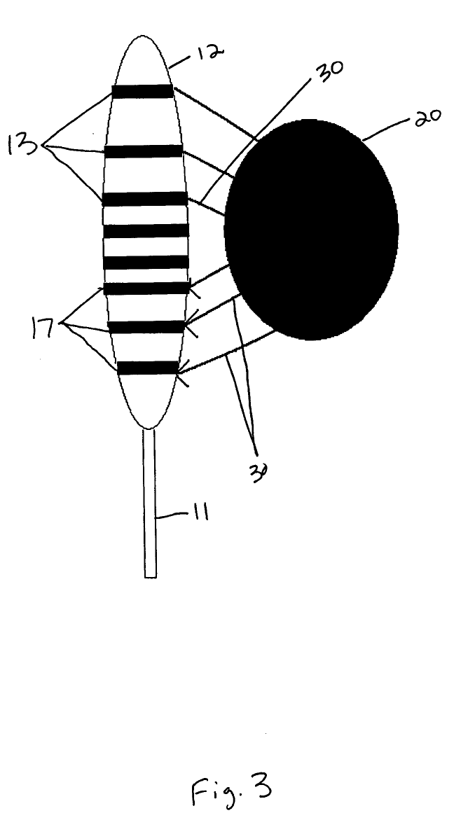

FIG. 3 is a perspective view of an embodiment of the present invention in which both electrode arrays are inserted into the body in proximity to the targeted tissue on a single expandable balloon. The system 10 comprises current injection electrodes 13 arranged in an array and attached to expandable balloon 12. Current return electrodes 17 are also arranged in an array and attached to expandable balloon 12. The expandable balloon 12 is removably attached to flexible tube 11, which is used to insert the balloon and electrode arrays inside the body in proximity to the targeted tissue 20. After expandable balloon 12 is inflated, a current generator, which is attached to the electrode arrays 13 and 17, is used to generate a current 30 that runs between the electrodes 13 and the electrodes 17 and runs through the targeted tissue 20. An image generator measures the resistivities of the targeted tissue 20 and creates an image of that tissue.

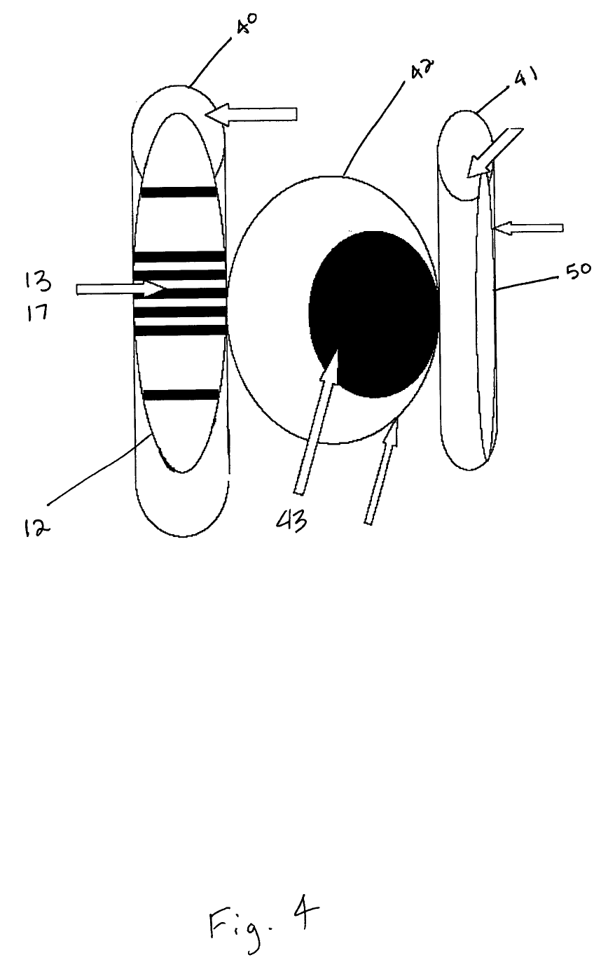

FIG. 4 is a perspective view of an embodiment of the present invention in which the system is used to create images of the prostate gland during treatment by a microwave antenna. In this embodiment, current injection electrodes 13 and current return electrodes 17 are arranged in arrays and attached to expandable balloon 12. The expandable balloon 12 is removably attached to a flexible tube, which is used to insert the balloon and electrode arrays inside the body through the rectum 40 in proximity to the prostate gland 42. A microwave antenna 50 is inserted into the body through the urethra 41 to treat the prostate gland 42. After expandable balloon 12 is inflated, a current generator, which is attached to the electrode arrays 13 and 17, is used to generate a current that runs between the electrodes 13 and the electrodes 17 and runs through the prostate gland 42. An image generator measures the resistivities of the prostate gland 42, including microwave heating pattern 43, and creates an image of that tissue.

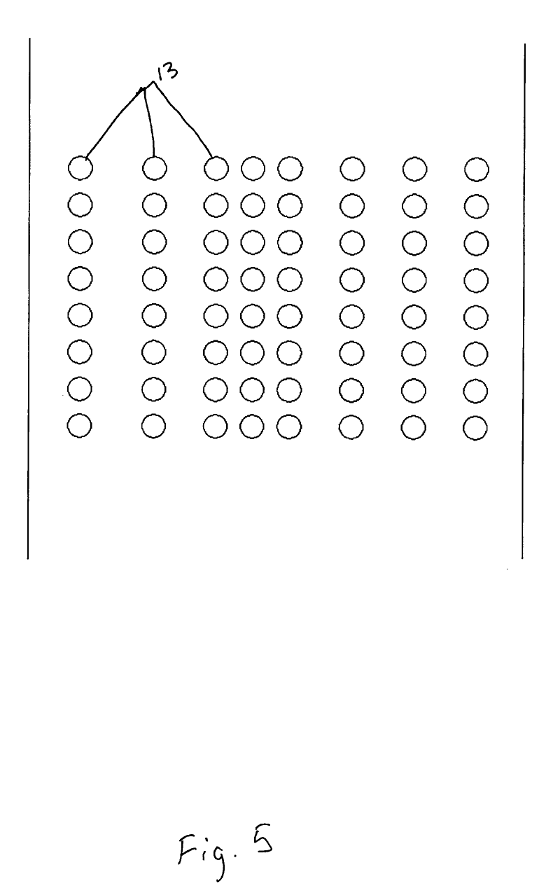

FIG. 5 is a representation of a possible array of electrodes 13 wrapped around a balloon surface. Those skilled in the art will understand that a variety of arrangements may be used to effectively inject current into the targeted tissue and receive such current.

In use, a user of on embodiment of the present invention would affix electrodes in an array to an expandable balloon in such a number and in such a pattern as to optimalize the image of the targeted body part. The user then would place the expandable balloon on a flexible tube, such as a catheter. The catheter then would be inserted in the body and advanced to the desired area, in proximity to the targeted tissue, through an appropriate entry point, such as a blood vessel, the rectum, or the urethra. The user would affix a second set of electrodes in an array either on the surface of the same expandable balloon, on the exterior surface of the body, or on a second expandable balloon which is inserted in the body and advanced to the targeted area in the same manner as the first balloon. The user then would use a current generator, such as a direct current generator or alternating current generator, which was attached to the electrodes to inject current into the targeted area. The user then would use an imaging device to calculate the resistivities of the tissues in the targeted area, and use algorithms to reconstruct the image of the targeted tissue and display or print it for the use of the user.

Although the present invention has been described with reference to preferred embodiments, persons skilled in the art will recognize that changes may be made in form and detail without departing from the spirit and scope of the invention.

What is claimed is:

1. A system for use in creating images of portions of human tissue inside the body by electrical impedance tomography, the system comprising:
at least one flexible tube for insertion in the body in proximity to a targeted portion of human tissue in the body;
an inflatable balloon removably attached to the distal end of the flexible tube;
a first array of electrodes attached to the surface of the inflatable balloon for at least one of injecting current into the targeted portion of human tissue and receiving current that was injected into the targeted portion of human tissue; and
a second array of electrodes for at least one of injecting current into the targeted portion of human tissue to the first array of electrodes and for receiving current from the first array of electrodes.

2. The system of claim 1 wherein the second array of electrodes is attached to the surface of the inflatable balloon.

3. The system of claim 1 wherein the second array of electrodes is attached to a second insertion device and inserted in the body in proximity to a targeted portion of human tissue in the body.

4. The system of claim 3 wherein the second insertion device further comprises:
a second flexible tube for insertion in the body in proximity to the targeted portion of human tissue and on an opposite side of the targeted tissue from the at least one flexible tube;
a second inflatable balloon removably attached to the distal end of the second flexible tube; and
wherein the second array of electrodes is attached to the surface of the second inflatable balloon.

5. The system of claim 1 wherein the second array of electrodes is attached to the outside of the body.

6. The system of claim 5 in which the second array of electrodes is mounted on a belt or girdle.

7. The system of claims 1, 2, 3, 4, 5, or 6, further comprising:
a current generator attached to the arrays of electrodes; and
an image generator attached to the arrays of electrodes and adapted to generate images of the targeted portion of human tissue.

8. A system for use in creating images of a prostate gland by electrical impedance tomography, the system comprising:
at least one flexible tube for insertion in the body in proximity to the prostate gland;
an inflatable balloon removably attached to the distal end of the flexible tube;
a first array of electrodes attached to the surface of the inflatable balloon for at least one of injecting current into the prostate gland and receiving current that was injected into the prostate gland; and
a second array of electrodes for at least one of injecting current into the prostate gland to the first array of electrodes and for receiving current from the first array of electrodes.

9. The system of claim 8 wherein the second array of electrodes is attached to the surface of the inflatable balloon.

10. The system of claim 8 wherein the second array of electrodes is attached to a second insertion device and inserted in the body in proximity to the prostate gland.

11. The system of claim 10 wherein the second insertion device further comprises:
a second flexible tube for insertion in the body in proximity to the prostate gland and on an opposite side of the prostate gland from the at least one flexible tube;
a second inflatable balloon removably attached to the distal end of the second flexible tube; and
wherein the second array of electrodes is attached to the surface of the second inflatable balloon.

12. The system of claim 8 wherein the second array of electrodes is attached to the outside of the body.

13. The system of claim 12 in which the second array of electrodes is mounted on a belt or girdle.

14. The system of claims 8, 9, 10, 11, 12, or 13, further comprising:
a current generator attached to the arrays of electrodes; and
an image generator attached to the arrays of electrodes and adapted to generate images of the targeted portion of human tissue.

15. A method for creating images of portions of human tissue inside the body by electrical impedance tomography, the method comprising:
inserting in the body in proximity to a targeted portion of tissue a first flexible tube with a first inflatable balloon removably attached to the distal end, such first inflatable balloon having first array of electrodes attached to its surface for at least one of injecting current into the targeted portion of human tissue and for receiving current that was injected into the targeted portion of human tissue;
placing in proximity to the targeted portion of tissue a second array of electrodes for at least one of injecting current into the targeted portion of human tissue to the first array of electrodes and for receiving current from the first array of electrodes;
injecting a current into the targeted portion of human tissue with the first or second array of electrodes; and
receiving the current with the second or first array of electrodes.

16. The method of claim 15 wherein the second array of electrodes is attached to the surface of the inflatable balloon.

17. The method of claim 15 wherein the second array of electrodes is attached to a second insertion device and inserted in the body in proximity to a targeted portion of human tissue in the body.

18. The method of claim 17 wherein the second insertion device comprises:
a second flexible tube for insertion in the body in proximity to the targeted portion of human tissue and on an opposite side of the targeted tissue from the at least one flexible tube;
a second inflatable balloon removably attached to the distal end of the second flexible tube; and
wherein the second array of electrodes is attached to the surface of the second inflatable balloon.

19. The method of claim 15 wherein the second array of electrodes is attached to the outside of the body.

20. The method of claim 19 in which the second array of electrodes is mounted on a belt or girdle.

21. The method of claims 15, 16, 17, 18, 19, or 20, further comprising the steps of:
determining the resistivities of the targeted portion of human tissue from the current readings; and
creating an image of the targeted portion of human tissue based on its resistivities.
